(19) United States
(12) Patent Application Publication
(10) Pub. No.: US 2020/0290989 A1
(43) Pub. Date: Sep. 17, 2020

(54) CALIXCROWNS AND USES THEREOF

(71) Applicant: INNOPHARMASCREEN INC., Incheon (KR)

(72) Inventors: Incheol KANG, Incheon (KR); Nari LEE, Incheon (KR)

(21) Appl. No.: 16/819,916

(22) Filed: Mar. 16, 2020

Related U.S. Application Data
(60) Provisional application No. 62/818,861, filed on Mar. 15, 2019.

(51) Int. Cl. C07D 323/00; G01N 33/553; G01N 33/68

(52) U.S. Cl. CPC C07D 323/00; G01N 33/6869; G01N 33/6845; G01N 33/553

(57) ABSTRACT

Provided herein are novel calixcrowns, such as those of Formula I, which are useful for coating a solid substrate such as a protein chip, diagnostic kit or protein separation pack. Also provided herein are methods detecting protein-protein interactions with a solid substrate coated with the calixcrown herein and an immobilized protein.

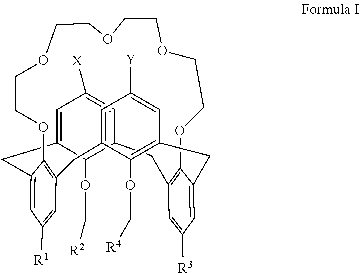

### CROSS-REFERENCE TO RELATED APPLICATIONS

This application claims benefit of the filing date of U.S. Appl. No. 62/818,861, filed Mar. 15, 2019, which is incorporated by reference herein in its entirety.

### BACKGROUND OF THE INVENTION

### Field of the Invention

In various embodiments, the present invention is generally related to novel calixcrowns and their uses in bioanalysis.

### Background Art

The immobilization of enzymes, antigens, antibodies and the like on solid carriers has become one of the most basic techniques in biotechnology and protein research, such as immunochemistry and enzyme chemistry. For example, the enzyme-linked immunosorbent assay (ELISA) is a technique that has been widely used in biotechnology for the assay of a particular protein or specific proteins causing a certain disease in experimental or clinical laboratories. Assay kits of such ELISA are commercially available in the market. More recently, development of protein chips, which require improved methods of protein immobilization on a solid matrix, is of a great concern in the field of biotechnology for the further advancement of proteomics research in the post-genomic era.

Previously, the immobilization of proteins such as antigens, antibodies or enzymes has been commonly practiced by physical adsorption of proteins on high molecular weight biopolymers such as various derivatives of collagen, dextran or cellulose. Covalent bonding between proteins and carrier surface by chemical reaction has been also widely used as a method for protein immobilization. The protein immobilization method by a “Sandwich” technique (triple-molecular layer) has been disclosed in literature Zhang, X. et al., Science 262:1706-1708 (1993), which describes a chemical bonding method by the biotin-avidin (or streptoavidin) interaction between proteins and carrier surface. That is, biotin is attached to the carrier surface and subsequently avidin or steptoavidin is linked thereto. Finally, proteins linked with biotin can be immobilized on the chemically modified carrier surface.

However, numerous problems are present in the various methods of protein immobilization as follows.

1. Density

The most critical problem of the protein immobilization method used in the past has been that the amount of protein immobilized on the surface of a substrate is extremely small. When the density of a protein to be immobilized on a carrier surface is low, other proteins may form non-specific binding. It is thus necessary to carry out a chemical treatment for the carrier surface to eliminate the undesired proteins bound to the carrier surface. However, such a chemical treatment may cause inactivation or denaturation of the immobilized protein molecule. In addition, even if a specific target protein is immobilized successfully onto the surface of a carrier, only an extremely small amount of the protein can be captured and consequently, the assay result may need to be further confirmed by other assay methods. When more protein is immobilized on a unit area on the surface of a carrier, the assay process is easier. In this regard, many studies have been carried out for the development of methods for a single molecular layer of proteins with the maximum amount immobilized on a carrier surface. A satisfactory result, however, has yet to be achieved.

2. Activity

In prior methods for protein immobilization by either chemical bonding or physical adsorption on surface of a carrier, the activity of an immobilized protein could be decreased in comparison with the free protein in a solution. It has been known that an immobilized protein on a solid carrier could lose its activity due to conformational changes or denaturation of the protein, especially around its active site as it binds tightly to the carrier surface via physical adsorption or chemical binding.

3. Orientation

In prior methods for protein immobilization on surface of a carrier, an active site of the protein may become essentially oriented toward the carrier surface in such a way that the active site is masked and thus the activity of the protein becomes lost. Such phenomena occurs in almost half of the immobilized proteins.

Calixcrowns were previously shown to be useful for forming a monolayer of calixcrowns on a solid substrate, which facilitates immobilization of a protein of interest, for example, through recognition of a cationic functional group of an amino acid on a protein surface, such as an ammonium group. See e.g., U.S. Pat. No. 6,485,984 B1 and Lee et al., Proteomics 3:2289-2304 (2003). WO2009069980A2 also describes various uses of calixcrowns in protein chip for determining kinase or phosphatase activity.

These earlier generations of calixcrowns can address the issues identified above to some extent. A need for new protein chips, for example, those with high sensitivity, still exists.

### BRIEF SUMMARY OF THE INVENTION

In various embodiments, the present disclosure is based at least in part on the discovery that certain novel calixcrowns can be used to coat a solid substrate, and the coated solid substrate can be used to immobilize a protein and to detect the presence of a protein (or a protein-protein interaction) in a sample with high sensitivity.

In some embodiments, the present disclosure is directed to a novel calixcrown having Formula I, II, or III, as defined herein. In some specific embodiments, the present invention is directed to compound 6 (IPS-Linker A) or IPS-Linker B as defined herein.

In some embodiments, a solid substrate coated with the calixcrown of the present disclosure is provided. In some embodiments, the solid substrate can be an inorganic or organic solid substrate, such as a substrate selected from the group consisting of gold, silver, glass, silicon, polystyrene, and polycarbonate. In some embodiments, the solid substrate is a protein chip, diagnostic kit or protein separation pack.

In some embodiments, a solid substrate with an immobilized protein is provided, wherein the solid substrate is coated with the calixcrown of the present disclosure. In some embodiments, the solid substrate can be an inorganic or organic solid substrate, such as a substrate selected from the group consisting of gold, silver, glass, silicon, polystyrene, and polycarbonate. In some embodiments, the immobilized protein can be antibodies, enzymes, membrane-bound receptors and non-membrane bound receptors, protein domains and motifs, and intracellular signaling proteins including modified proteins, such as, e.g., bromodomain-containing protein 4, heparing binding domain, Polo-Box Domain, etc.

In some embodiments, the present disclosure provides a method of immobilizing a protein on a solid substrate. Typically, the method can comprise applying the calixcrown of the present disclosure onto an inorganic or organic solid substrate to form a calixcrown coated solid substrate; and subsequently immersing the calixcrown coated solid substrate into a solution comprising the protein.

In some embodiments, a method of detecting protein-protein interaction is also provided. For example, in some embodiments, the method can comprise immobilizing a first protein on a solid substrate coated with the calixcrown of the present disclosure to form a solid substrate with immobilized first protein; incubating the solid substrate with immobilized first protein with a solution comprising a second protein; and detecting the interaction between the immobilized first protein and the second protein. In some embodiments, the second protein is a biomarker for a disease.

### BRIEF DESCRIPTION OF THE DRAWINGS/FIGURES

FIG. 1A. A graph showing the fluorescence signal detected from a ProLinker coated slide immobilized with Aβ₁₋₄₂ and incubated with various concentration of VEGF₁₆₅.

FIG. 1B. A graph showing the fluorescence signal detected from an IPS-Linker coated slide immobilized with Aβ₁₋₄₂ and incubated with various concentration of VEGF₁₆₅.

FIG. 2. Human IL-17 detection using IPS-Linker coated chip.

FIG. 3. A graph showing the results of a protein-protein interaction assay for IL-23 and its receptor using IPS-Linker-coated chip.

FIG. 4. A graph showing the results of a protein-protein interaction assay for PD-1 and PD-L1 using IPS-Linker-coated chip.

FIG. 5. A graph showing the results of a protein-nucleotide interaction assay for STING and c-di-GMP binding using IPS-Linker-coated chip.

### DETAILED DESCRIPTION OF THE INVENTION

In various embodiments, the present disclosure provides novel calixcrowns and their uses in bioanalysis.

### Calixcrowns

Calixcrowns were discovered to have ionophoric properties toward alkali and alkaline earth metal cations and also to tertiary amines. Their binding selectivity is greatly determined by the number of oxygen atoms in the polyethylene glycol bridge, the nature of the substituents at the crown bridge, and also by the stereochemistry of the calixarene skeleton at the binding site. See generally, Salorinne et al., J. Incl. Phenom. Macrocyc.l Chem. 61:11-27 (2008), incorporated herein by reference in its entirety.

The calixcrowns of the present disclosure are typically calix[4]crowns. Calix[4]crowns can be 1,3- or 1,2-bridged. The calix[4]crowns of the present disclosure are typically 1,3-bridged. Typically, the calixcrowns of the present disclosure can adopt a 1,3-alternate conformation, although in some cases, a partial cone or cone conformation can also be possible.

The calixcrowns of the present disclosure can typically have a structure according to Formula I, II, or III. In some embodiments, the calixcrowns herein can have an amino group and/or a carboxylic acid group, and such calixcrowns can exist in the form of a salt. For calixcrowns herein having a carboxylic acid function, their esters, such as C₁₋₄ alkyl esters, are also novel compounds of the present disclosure. These esters can be useful, for example, as intermediates for preparing compounds with the corresponding carboxylic acid function.

In some embodiments, the calixcrown of the present disclosure can be characterized by a Formula I:

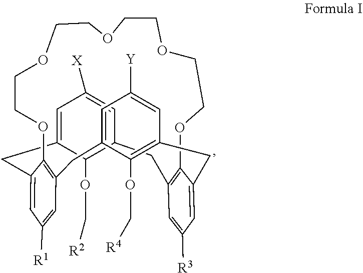

wherein:
R¹ and R³ independently represent hydrogen, —CH₂SH, —CH₂Cl, —CH₂CN, —CH₂CHO, —CH₂NH₂, or —CH₂COOH;
R² and R⁴ independently represents —CH₂SH, —CH₂Cl, —CH₂CN, —CH₂CHO, —CH₂NH₂, —CH₂COOH, —CN, —CHO, or —COOH; and
X and Y independently represent hydrogen, C₁₋₄ alkyl, OH, or C₁₋₄ alkoxy.

Typically, in Formula I, R¹ and R³ are hydrogen. However, in some embodiments, R¹ and R³ can also independently be a group that has affinity to the surface of a solid substrate, such as gold. In some embodiments, R¹ and R³ can also be independently a group such as —CH₂SH, —CH₂CHO, —CH₂NH₂, or —CH₂COOH.

Typically, in Formula I, X and Y are hydrogen. However, in some embodiments, X and Y can also independently be a group such as C₁₋₄ alkyl, OH, or C₁₋₄ alkoxy.

Typically, in Formula I, R² and R⁴ are —COOH. In some embodiments, R² and R⁴ can also independently be a group that has affinity to the surface of a solid substrate, such as gold. In some embodiments, R² and R⁴ can also be independently a group such as —CH₂SH, —CH₂CHO, —CH₂NH₂, —CHO, or —CH₂COOH.

In some embodiments, the calixcrown of Formula I is compound 6 (IPS-Linker A) or IPS-Linker B:

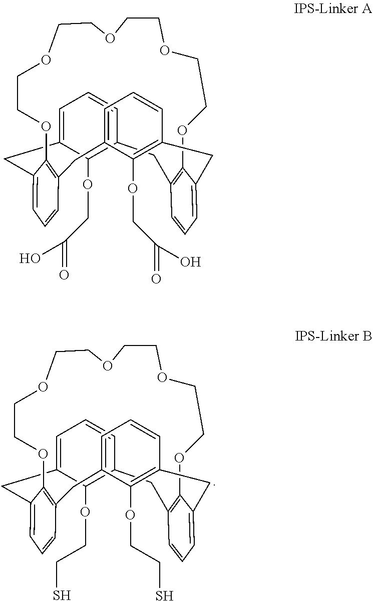 [SMILES: O=C(O)COc1c2cccc1Cc1cccc3c1OCCOCCOCCOCCOc1c(cccc1C2)CC1=CC=CC(C3)C1OCC(=O)O ; SCCOc1c2cccc1Cc1cccc3c1OCCOCCOCCOCCOc1c(cccc1C2)CC1=CC=CC(C3)C1OCCS]

In some embodiments, the calixcrown of the present disclosure can be characterized by a Formula II:

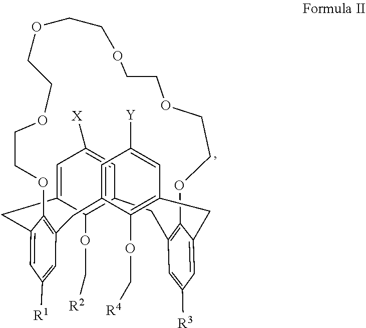

wherein:
R¹ and R³ independently represent hydrogen, —CH₂SH, —CH₂Cl, —CH₂CN, —CH₂CHO, —CH₂NH₂, or —CH₂COOH;
R² and R⁴ independently represents —CH₂SH, —CH₂Cl, —CH₂CN, —CH₂CHO, —CH₂NH₂, —CH₂COOH, —CN, —CHO, or —COOH; and
X and Y independently represent hydrogen, C₁₋₄ alkyl, OH, or C₁₋₄ alkoxy.

Typically, in Formula II, R¹ and R³ are hydrogen. However, in some embodiments, R¹ and R³ can also independently be a group that has affinity to the surface of a solid substrate, such as gold. In some embodiments, R¹ and R³ can also be independently a group such as —CH₂SH, —CH₂CHO, —CH₂NH₂, or —CH₂COOH.

Typically, in Formula II, X and Y are hydrogen. However, in some embodiments, X and Y can also independently be a group such as C₁₋₄ alkyl, OH, or C₁₋₄ alkoxy.

Typically, in Formula II, R² and R⁴ are —COOH. In some embodiments, R² and R⁴ can also independently be a group that has affinity to the surface of a solid substrate, such as gold. In some embodiments, R² and R⁴ can also be independently a group such as —CH₂SH, —CH₂CHO, —CH₂NH₂, —CHO, or —CH₂COOH.

In some embodiments, the calixcrown of the present disclosure can be characterized by a Formula III:

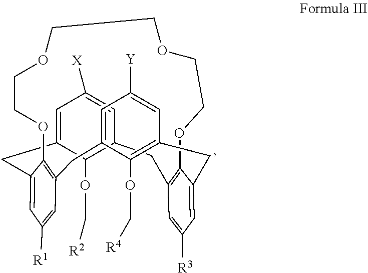

wherein:
R¹ and R³ independently represents hydrogen, —CH₂SH, —CH₂Cl, —CH₂CN, —CH₂CHO, —CH₂NH₂, or —CH₂COOH;
R² and R⁴ independently represents —CH₂SH, —CH₂Cl, —CH₂CN, —CH₂CHO, —CH₂NH₂, —CH₂COOH, —CN, —CHO, or —COOH; and
X and Y independently represent hydrogen, C₁₋₄ alkyl, OH, or C₁₋₄ alkoxy.

Typically, in Formula III, R¹ and R³ are hydrogen. However, in some embodiments, R¹ and R³ can also independently be a group that has affinity to the surface of a solid substrate, such as gold. In some embodiments, R¹ and R³ can also be independently a group such as —CH₂SH, —CH₂CHO, —CH₂NH₂, or —CH₂COOH.

Typically, in Formula III, X and Y are hydrogen. However, in some embodiments, X and Y can also independently be a group such as C₁₋₄ alkyl, OH, or C₁₋₄ alkoxy.

Typically, in Formula III, R² and R⁴ are —COOH. In some embodiments, R² and R⁴ can also independently be a group that has affinity to the surface of a solid substrate, such as gold. In some embodiments, R² and R⁴ can also be independently a group such as —CH₂SH, —CH₂CHO, —CH₂NH₂, —CHO, or —CH₂COOH.

The calixcrowns disclosed herein can be readily prepared by those skilled in the art in view of this disclosure. For example, the calixcrowns can easily be prepared by the reaction of calix[4]arene with the appropriate polyethylene glycol ditosylate in the presence of various bases. An example of preparation of the calixcrown herein (compound 6) is also detailed in the Examples section.

### Biochips

The calixcrowns of the present disclosure are typically bifunctional compounds, which allow them to bind to a surface and recognize a cationic functional group. The calixcrowns of the present disclosure can also typically self-assemble to form a monolayer on solid substrates, which can capture substances, such as proteins, with a cationic functional group through the crownether moiety.

Accordingly, in various embodiments, the present disclosure also provides solid substrates coated with the calixcrowns of the present disclosure, methods of preparing the same, and various associated uses. In some embodiments, the solid substrate can be a protein chip, diagnostic kit, or a protein separation pack. In some embodiments, the solid substrate can also be a well-on-a-chip, an array, etc. which can be used, e.g., in high throughput analysis/screening.

In some embodiments, the present disclosure provides a solid substrate coated with the calixcrown of the present disclosure (e.g., compound 6). In some embodiments, the solid substrate can be an inorganic or organic solid substrate. In some embodiments, the solid substrate can be an inorganic solid substrate. Typically, the inorganic solid substrate can be a metal or glass substrate. For example, in some embodiments, the solid substrate can be a metal substrate such as gold, silver, platinum, etc. In some embodiments, the solid substrate can also be glass substrate. In some embodiments, the solid substrate can be a polymer based substrate. Non-limiting examples include polystyrene and polycarbonate substrates. In some embodiments, the solid substrate can be selected from the group consisting of gold, silver, glass, silicon, polystyrene, and polycarbonate. In any of the embodiments described herein, the calixcrowns of the present disclosure can form a monolayer on the solid substrate.

Methods for coating the solid substrate with the calixcrowns of the present disclosure are described herein. Typically, the methods comprise applying the calixcrown of the present disclosure onto the solid substrate.

In some embodiments, the present disclosure also provides a solid substrate with immobilized protein, wherein the solid substrate is coated with the calixcrown of the present disclosure (e.g., as described hereinabove, such as compound 6). In some embodiments, the solid substrate can be an inorganic or organic solid substrate, such as a substrate selected from the group consisting of gold, silver, glass, silicon, polystyrene, and polycarbonate. In some embodiments, the immobilized protein can be antibodies, enzymes, membrane-bound receptors and non-membrane bound receptors, protein domains and motifs, and intracellular signaling proteins including modified proteins, such as, e.g., bromodomain-containing protein 4, heparin binding domain, Polo-Box Domain, etc.

Methods for immobilizing a protein on the solid substrate are described herein. For example, in some embodiments, the method can comprise applying the calixcrown of the present disclosure (e.g., compound 6) onto an inorganic or organic solid substrate to form a calixcrown coated solid substrate; and subsequently immersing the calixcrown coated solid substrate into a solution comprising the protein. Typically, the calixcrowns of the present disclosure form a monolayer on the solid substrate. The solution can be a buffer solution. After immersing the calixcrown coated solid substrate into the protein solution, the mixture can be incubated for a period of time to allow the protein to interact with the calixcrown. Typically, the solution contains the protein in a concentration of about 1 nM to about 500 uM, preferably, about 1 uM to about 500 uM, such as about 50 uM to about 250 uM, or any concentration up to the solubility limit of the protein. Suitable solid substrates are described herein. After incubation with the protein solution, the solid substrate is typically washed, blocked to remove non-specific bindings, and dried. Exemplary procedures are detailed in the Examples section.

The solid substrates with an immobilized protein herein can be used further for detection of protein-protein interactions or for detecting a biomarker. For example, in some embodiments, the present disclosure provides a method of detecting protein-protein interaction, the method can comprise immobilizing a first protein on a solid substrate coated with the calixcrown of the present disclosure to form a solid substrate with immobilized first protein; incubating the solid substrate with immobilized first protein with a solution comprising a second protein; and detecting the interaction between the immobilized first protein and the second protein. In some embodiments, the second protein can be a biomarker, for example, antibodies, enzymes, membrane-bound receptors and non-membrane bound receptors, protein domains and motifs, and intracellular signaling proteins including modified proteins, such as, e.g., bromodomain-containing protein 4, heparin binding domain, Polo-Box Domain, etc. In some embodiments, the solution comprising the second protein derives from a biological fluid sample from a patient, such as a human patient. In some embodiments, the patient can have cancer and the second protein is a biomarker for the cancer. In some embodiments, the biological fluid sample can be a raw sample, diluted sample, or otherwise processed sample. In some embodiments, the solution can have a concentration of the second protein ranging from about 1 aM (atto M) to about 100 uM, for example, about 1 fM (femto M) to about 10 uM, about 10 fM to about 10 uM, from about 50 fM to about 2 uM, from about 100 fM to about 1 uM, from about 250 fM to about 1 uM, etc. For example, the protein can be detected by a colorimetric enzyme assay used in the ELISA system.

A typical ELISA system method includes:

Plate Preparation

Dilute the Capture Antibody to the working concentration in PBS (1×) without carrier protein. Immediately coat a 96-well microplate with 100 μL per well of the diluted Capture Antibody. Seal the plate and incubate overnight at room temperature. Aspirate each well and wash with Wash Buffer (PBS 1× containing 0.05% Tween 20), repeating the process two times for a total of three washes. Wash by filling each well with Wash Buffer (400 μL) using a squirt bottle, manifold dispenser, or autowasher. Complete removal of liquid at each step is essential for good performance. After the last wash, remove any remaining Wash Buffer by aspirating or by inverting the plate and blotting it against clean paper towels. Block plates by adding 300 μL of Reagent Diluent (PBS 1× containing 1% BSA) to each well. Incubate at room temperature for a minimum of 1 hour. Repeat the aspiration/wash as in step 2. The plates are now ready for sample addition.

Assay Procedure

Add 100 μL of sample or standards in Reagent Diluent, or an appropriate diluent, per well. Cover with an adhesive strip and incubate 2 hours at room temperature. Repeat the aspiration/wash as in step 2 of Plate Preparation. Add 100 μL of the Detection Antibody, diluted in Reagent Diluent, to each well. Cover with a new adhesive strip and incubate 2 hours at room temperature. Repeat the aspiration/wash as in step 2 of Plate Preparation. Add 100 μL of the working dilution of Streptavidin-HRP to each well. Cover the plate and incubate for 20 minutes at room temperature. Avoid placing the plate in direct light. Repeat the aspiration/wash as in step 2. Add 100 μL of Substrate Solution (1:1 mixture of Color Reagent A (H2O2) and Color Reagent B (Tetramethylbenzidine) (R&D Systems, Minneapolis, Minn.) to each well. Incubate for 20 minutes at room temperature. Avoid placing the plate in direct light. Add 50 μL of Stop Solution (2 N H2SO4 (R&D Systems, Minneapolis, Minn.) to each well. Gently tap the plate to ensure thorough mixing. Determine the optical density of each well immediately, using a microplate reader set to 450 nm. If wavelength correction is available, set to 540 nm or 570 nm. If wavelength correction is not available, subtract readings at 540 nm or 570 nm from the readings at 450 nm. This subtraction will correct for optical imperfections in the plate. Readings made directly at 450 nm without correction may be higher and less accurate.

As described in the Examples section, the solid substrate coated with the calixcrown of the present disclosure (e.g., Compound 6) and subsequently an immobilized protein (Aβ₁₋₄₂) was able to detect VEGF₁₆₅ at a much lower concentration when compared to a control solid substrate coated with an earlier generation of calixcrown, ProLinker. See U.S. Pat. No. 6,485,984 B1 and Lee et al., Proteomics 3:2289-2304 (2003), each of which is incorporated herein by reference in its entirety. WO2009069980A2 also describes various uses of calixcrowns in protein chip for determining kinase or phosphatase activity, incorporated herein by reference in its entirety. Thus, using the calixcrown of the present disclosure can improve detection limits and sensitivities, which are advantageous over existing techniques.

Various methods for detecting the second protein captured on the solid substrate can be used. For example, in some embodiments, the detecting comprises contacting an antibody for the second protein with the solid substrate. In some embodiments, the detecting further comprises contacting a dye-labeled secondary antibody with the solid substrate, wherein the secondary antibody binds to the antibody for the second protein. In some embodiments, the detecting further comprises measuring a level of the dye-labeled secondary antibody associated with the solid substrate. Any suitable dye can be used. Typically, the dye is a fluorescent dye and the measuring comprises measuring the fluorescent signals. For example, in some embodiments, the dye can be Cy5.

### Definitions

It is meant to be understood that for the formulae herein, proper valences are maintained for all moieties and combinations thereof.

It is also meant to be understood that a specific embodiment of a variable moiety herein can be the same or different as another specific embodiment having the same identifier.

Suitable groups for the variables in compounds of Formula I, II, or III, as applicable, are independently selected. The described embodiments of the present invention can be combined. Such combination is contemplated and within the scope of the present invention. For example, definitions of one of the variables can be combined with any of the definitions of any other of the variables in Formula I, II, or III.

As used herein, the term “calixcrown(s) of the present disclosure” or the like refers to any of the compounds described herein according to Formula I, II, or III, or Compound 6, isotopically labeled compound(s) thereof, tautomers thereof, conformational isomers thereof, salts thereof (e.g., base addition salt such as Na salt) and/or esters thereof (e.g., C₁₋₄ alkyl ester).

As used herein, the term “alkyl” as used by itself or as part of another group refers to a straight- or branched-chain aliphatic hydrocarbon, typically has 1-20 carbons. In some embodiments, the alkyl group is a straight chain C₁₋₆ alkyl group. In other embodiments, the alkyl group is a branched chain C₃₋₆ alkyl group. In other embodiments, the alkyl group is a straight chain C₁₋₄ alkyl group. As understood by those skilled in the art, an alkyl group is saturated. A C₁₋₄ alkyl group as used herein refers to a methyl, ethyl, propyl, isopropyl, butyl, sec-butyl, tert-butyl, or iso-butyl. As also understood by those skilled in the art, an “alkylene” group refers to a divalent radical derived from a corresponding alkyl group. For example, a C₁₋₄ alkylene group as used herein refers to methylene, ethylene, propylene, isopropylene, butylene, sec-butylene, tert-butylene, or iso-butylene.

As used herein, the term “alkoxy” as used by itself or as part of another group refers to a radical of the formula OR^{a1}, wherein R^{a1} is an alkyl.

The term “patient” as used herein, refers to an animal, such as a mammal, a nonhuman, or human, who has been the object of treatment, observation or experiment.

### Examples

### Example 1. Preparation of Calixcrown 6

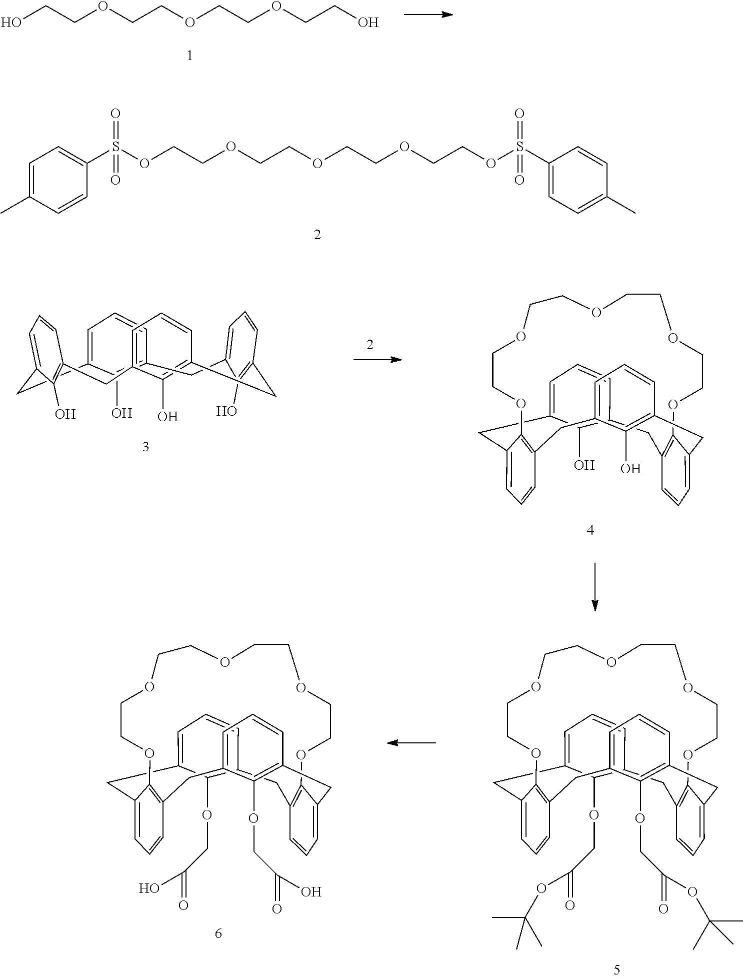

### 1) Synthesis of Compound 1 (Tetraethylene glycol ditosylate)

Tetraethylene glycol (4 mL, 23 mmol) was dissolved in anhydrous chloroform (30 mL). The solution was cooled to −20° C. in a sodium chloride ice bath. Tosyl chloride (13 g, 69 mmol) and anhydrous pyridine (24 mL) ware added sequentially while keeping the temperature of the solution below 0° C. After 5 h reaction at −20° C., chloroform and pyridine were removed under reduce pressure, ice water (250 mL) was added and the solution was extracted with CH₂Cl₂ (200 mL, each) three times. The combined organic phase was washed twice with HCl (2N, 250 mL) and water (200 mL) sequentially. After the organic layer was dried over sodium sulfate the solvent removed under reduced pressure. The residue was subjected to silica chromatography using ethyl acetate/hexane (in a ratio of 2:8 to 5:5 of ethyl acetate:hexane) to give product in 725 yield (9.45 g) as coolness oil. R_{f}=0.31 (silica, 1:1, ethyl acetate/hexane). ¹H NMR (400 MHz, CDCl₃) δ ppm 2.45 (s, 6H), 3.68 (t, J=4.7 Hz, 4H), 4.16 (t, J=4.7 Hz, 4H), 7.35 (d, J=8.2 Hz, 4H), 7.80 (d, J=8.2 Hz, 4H), ¹³C NMR (400 MHz, CDCl₃) δ ppm 21.6, 68.7, 69.3, 70.5, 70.7, 128.0, 129.8, 133.0, 144.8.

### 2) Synthesis of Compound 2

2-1) p-tert-Butyl phenol (compound 1) (150 g, 1 mol) and NaOH (1.8 g, 45 mmol) was dissolved in 37% formaldehyde (100.7 g, 1.24 mol). The reaction mixture was refluxed at 120° C. for 12 h. After the solution was cooled to room temperature, H₂O was removed in vacuo, and then, diphenyl ether (450 mL) and toluene (150 mL) were added. The reaction mixture was again refluxed at 250° C. The color of the reaction mixture changed to dark brown. Then, the crude product was recrystallized from ethyl acetate (300 mL) and was washed with acetic acid (100 mL). White crystalline solid 2 in 56.79% yield. ¹H NMR (400 MHz, CDCl₃) δ ppm 10.36 (s, 4H), 7.04 (s, 8H), 4.25 (d, 4H), 3.49 (d, 4H), 1.21 (s, 36H).

### 3) Synthesis of Compound 3

2-2) p-tert-butylcalix[4]arene (compound 2) (10.00 g, 13.5 mmol), toluene (100 mL) and phenol (1.75 g, 18.60 mmol) were added to a flask and the solution was stirred under argon for 10 min. With vigorous mechanical stirring, aluminum trichloride (10.00 g, 75.0 mmol) was added. The mixture was stirred at room temperature for 5 h. The mixture was poured into a 500-mL beaker containing crushed ice (200 g) and extracted with CH₂Cl₂ (400 mL). The organic layer was washed with 1 N HCl (3×100 mL) and water (2×100 mL) and dried over NaSO₄. The solvent was evaporated in vacuo. Diethyl ether (50 mL) was added to the oily orange residue and the heterogeneous mixture was kept at −15° C. for 1 h. The precipitated solid was filtered and triturated with diethyl ether (100 mL). The mixture was kept at −15° C. for 1 h and filtered to provide. 5.54 g (90%) of light, yellow powder. ¹H NMR (400 MHz, CDCl₃): δ 10.20 (s, 4H), 7.04 (d, 8H, J=7.6 Hz), 6.73 (t, 4H, J=7.6 Hz), 4.24 (br s, 4H), 3.54 (br s, 4H).

### 3) Synthesis of Compound 4

To a mixture of NaH (5.00 eq, 2.16 g, 90.0 mmol) and DMF (1300 mL) in a 2000 mL, three-necked flask under nitrogen was added a solution of 25,26,27,28-tetrahydroxycalix[4]arene (compound 3) (1.00 eq, 7.64 g, 18.0 mmol) in DMF (100 mL) over a 1 h period. The mixture was stirred for an additional hour. Tetraethylene glycol ditosylate (2.20 eq, 13.88 g, 39.6 mmol) in DMF (100 mL) was added over a 1 hr period. The mixture was stirred at 50° C. for 72 hr. The reaction was quenched by addition of H₂O (50 mL), and the solution was extracted with CH₂Cl₂ (50 mL) three times. The combined organic phase was washed twice with HCl (3N, 50 mL) and water (200 mL) sequentially. After the organic layer was dried over sodium sulfate, the solvent was removed under reduced pressure. The residue was subjected to silica chromatography using ethyl acetate/hexane (in a ratio of 1:4 to 1:2 of ethyl acetate:hexane) to give product in 45% yield (2.34 g) as light yellow powder. R_{f}=0.55 (silica, 1:1, ethyl acetate/hexane). ¹H NMR (400 MHz, CDCl₃): δ 8.34 (s, 2H), 7.13-6.87 (m, 8H), 6.80 (t, J=7.5, 2H), 6.60 (t, J=7.5, 2H), 4.62-4.41 (m, 12H), 4.41-4.28 (m, 2H), 4.11 (t, J=9.0, 2H), 4.06-3.88 (m, 6H), 3.87-3.65 (m, 4H), 3.39 (d, J=12.3, 1H), 3.36 (d, J=13.5, 2H), 3.32 (d, J=3.8, 1H).

### 4) Synthesis of Compound 5

In a round bottom flask, compound 4 (1 g, 1.77 mmol) was taken in dry acetonitrile. Potassium carbonate (1.17 g, 8.41 mmol) and Tert-butyl bromoacetate (0.90 g, 4.61 mmol) was added and mixture was refluxed 24 hrs. After completion, solvent evaporated, water was added (100 ml) extracted with dichloromethane (2×100 mL). Organic layer separated, dried over sodium sulfate, filtered and concentrated. Water (250 mL) was added, and the solution was extracted with CH₂Cl₂ (200 mL, each) three times. The residue was subjected to silica chromatography using ethyl acetate/hexane (in a ratio of 1:3 of ethyl acetate:hexane) to give product in 90% yield (1.1 g) as light yellow powder. R_{f}=0.6 (silica, 1:2, ethyl acetate/hexane). ¹H NMR (400 MHz, CDCl₃): δ 6.59 (m, 12H), 4.64 (d, 4H), 4.21 (m, 2H), 3.98 (m, 6H), 3.18 (m, 4H), 1.45 (s, 18H).

### 5) Synthesis of Compound 6 (IPS Linker A)

An aqueous solution (252 mL) of NaOH (15%) was added to an ethanolic solution (70 mL) of compound 5 (1.16 g, 1.68 mmol). The reaction mixture was heated under reflex after 24 hr. It was cooled to room temperature, and the solvent was removed by rotary evaporation. Then 50 mL of cold water was added to the solid mass and HCl (5 N) was added dropwise with vigorous stirring until the pH of the solution reached 7. The solution was extracted with CH₂Cl₂ (50 mL) three times. The combined organic phase was washed twice with water (50 mL) sequentially. After the organic layer was dried over sodium sulfate the solvent removed under reduced pressure. The residue was subjected to silica chromatography using methanol/methylene chloride (in a ratio of 1:9 to 1:1 of methanol:methylene chloride) to give product in 60% yield (0.7 g) as light yellow powder. R_{f}=0.3 (silica, 3:1, methanol:methylene chloride). ¹H NMR (300 MHz, CDCl₃): 7.08 (m, 8H), 6.80 (s, 4H), 4.43 (m, 4H), 4.22-3.66 (m, 16H), 3.35 (m, 4H).

### Example 2. Preparation of Protein Chip Coated with Calixcrown 6

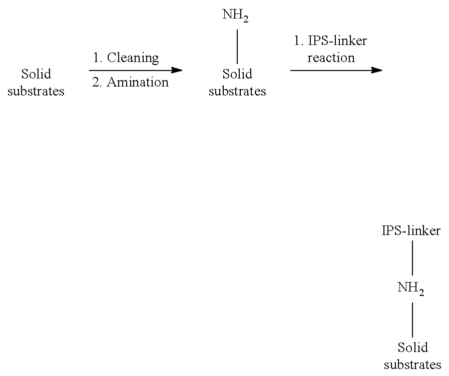

Slide glass was placed in 60 mL of the cleaning solution (Methanol:35% hydrogen chloride=1:1) and washed for 30 minutes. The slide was soaked in Piranha solution (surfuric acid:hydrogen peroxide=3:1) and washed with distilled water for 30 minutes. The slide glass dried with nitrogen gas was soaked in 60 ml of the amination solution (3% (3-Aminopropyl) triethoxysilane in Ethanol) for 2 hrs under the dark condition. It was rinsed three times with ethanol followed by distilled water, repeatedly, and then finally washed with ethanol. The slide was dried with nitrogen gas and react at 100° C. for 2 hrs. It was soaked with 60 ml of A solution (10 mg IPS-linker (Calixcrown 6) in DMF, 5 mg N-(3-dimethylaminopropyl)-N′-ethylcarbodiimide hydrochloride (EDC-HCl), 3 mg 1-hydroxybenzotriazole hydrate (HOBt), 1 mg 4-N,N-dimethylaminopyridine(DMAP)) and incubated for 12 hrs at room temperature. The slide was washed 3 times with 70 ml of DMF for 10 min and repeated 2 more times. The slide was dried with nitrogen gas and stored at room temperature under vacuum until use.

The structure of IPS-linker (compound 6) is as follows:

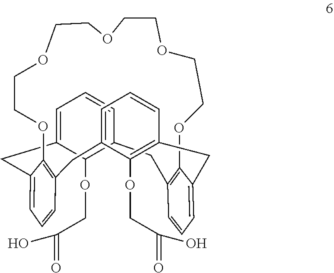 [SMILES: O=C(O)COc1c2cccc1Cc1cccc3c1OCCOCCOCCOCCOc1c(cccc1C2)CC1=CC=CC(C3)C1OCC(=O)O]

Preparation of ProteoChip Coated with Prolinker

Glass slides are immersed in cleaning solution (methanol:HCl=1:1) for 30 minutes and Piranha solution for 10 minutes (room temperature). Wash thoroughly with Sterile D.W. and dry. Immerse the glass slides in the amination solution for 6 hours at room temperature. After washing with toluene, washing with absolute ethanol. Incubation at 100° C. for 1 hour. Aminated glass slides were immersed in 10 mM Prolinker in Chloroform solution for 3 hours at room temperature. After washing with chloroform, washing with absolute ethanol. After washing with Sterile D.W., completely dry.

Aβ₁₋₄₂ in a dilution buffer (30% glycerol in PBS, pH 7.4,) was spotted onto the ProteoChip (Proteogen, Korea, ProLinker coated chip) or IPS-CHIP (Innopharmascreen, Korea, IPS-linker coated chip), and the chips were incubated in a humidity chamber at 4° C. overnight. The structure of the prolinker for the ProteoChip follows:

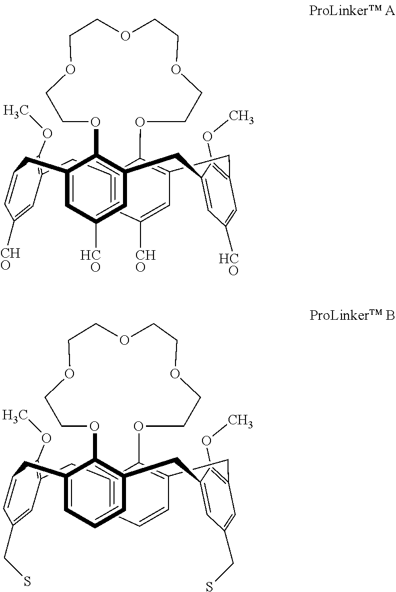

The chips were rinsed in PBST (0.5% Tween-20 in PBS) two times for 10 min and incubated with 3% BSA containing a 0.05% Tween-20 solution at room temperature for blocking nonspecific binding. After extensive rinsing, the Aβ₁₋₄₂ microarray can be used to detect protein interactions with Aβ₁₋₄₂.

### Example 3. Protein-Protein Interaction Assay of VEGF-Aβ Binding Using Protein Chip

The Aβ₁₋₄₂ microarray prepared in Example 2 were spotted with a mixture of a VEGF₁₆₅ (Vexxon, Korea). After rinsing with PBST and DW, rabbit-anti-VEGF (A20) (Santacruz, Germany) diluted to 1:10 with 3% BSA and 30% glycerol in PBS was spotted for recognition of a VEGF₁₆₅ bound to Apr-42. After rinsing with PBST and DW, anti-rabbit secondary antibody labeled with Cy5 (Invitrogen, USA) which was diluted to 1:100 with PBS containing 3% BSA and 30% glycerol, was applied on the chip at 30° C. for 1 hr. After rinsing with PBST and DW, the chips were dried in a stream of N₂ gas. The protein-protein interaction was determined by measuring relative fluorescence intensity of the mixture spot versus the control spot (VEGF alone).

Detection and Data Analysis:

The chip was scanned using a Genetix aQuire™ scanner (Genetix, UK) and saved as a TIFF file. The scanned images were analyzed using a GenePix Pro 6.0 (Axon Instruments, CA, USA), and the data analyzed with Excel (Microsoft, Redmond, Wash.) and Origin 6.1 (Originlab, MA, USA).

Results:

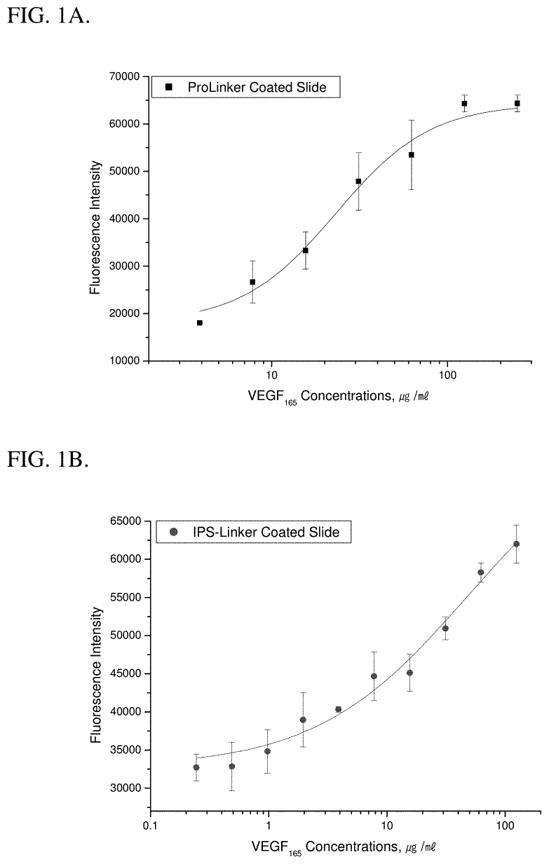

The Aβ microarray was constructed for analysis of Aβ1-42-VEGF165 interaction. To construct Aβ microarrays, soluble Aβ1-42 (50 mg/ml) was immobilized as a capture molecule on each protein chip base plate. Aβ1-42 microarray on the chip was interacted with VEGF165 in different concentrations ranged from 0.25 to 250.0 μg/mL (FIGS. 1A and 1B). It was shown that the Aβ1-42 interacted well with VEGF165 in a dose-dependent manner in both chip systems. As shown in the figure, IPS-CHIP coated with IPS-Linker was able to detect a low level (0.25 mg/ml) of VEGF protein compared to ProteoChip coated with ProLinker (approx. 3.9 mg/ml). Taken together, this finding demonstrated that IPS-Linker-coated chip was more sensitive than ProLinker-coated chip in terms of detecting protein.

### Example 4. Human IL-17 Detection by Using IPS-Linker-Coated Chip

The IL-17 capture antibody (R&D systems, USA) was immobilized on IPS-Linker-coated chip for overnight at 4° C. The chip was rinsed in washing solution twice for 10 minutes each, and then blocked with blocking solution for 1 hr. The blocked IL-17 antibody chip was rinsed in washing solution and DW (distilled water), then dried under a N₂ stream. After extensive rinsing, the recombinant IL-17 protein (R&D systems, USA) in the reaction solution was spotted on the IL-17 antibody-chip for 2 hr in a humidity chamber at 37° C. The chip was rinsed in washing solution and DW, then dried under a N₂ stream. After extensive rinsing, the IL-17 detection antibody (R&D systems, USA) in the reaction solution was spotted on the chip and incubated with for 1 hr in a humidity chamber at 37° C. Then the chip was rinsed in washing solution and DW, followed by being dried under a N₂ stream. After extensive rinsing, Cy5 labeled Streptavidin (GE Healthcare, USA) in the reaction solution was spotted on the chip and incubated for 1 hr in a humidity chamber at 37° C. Following rinsing with PBST (PBS-Tween 20) and DW, the chip was dried under a N₂ stream, and the fluorescence intensity was measured using a fluorescence scanner.

Detection and Data Analysis:

The chip was scanned by using a GenePix 4000B scanner (Molecular Devices, USA) and saved as TIFF files. The scanned images were analyzed using a GenePix Pro 6.0 (Molecular Devices, USA), and the data analyzed with Excel (Microsoft, WA) and Origin 6.1 (Originlab, USA).

Results:

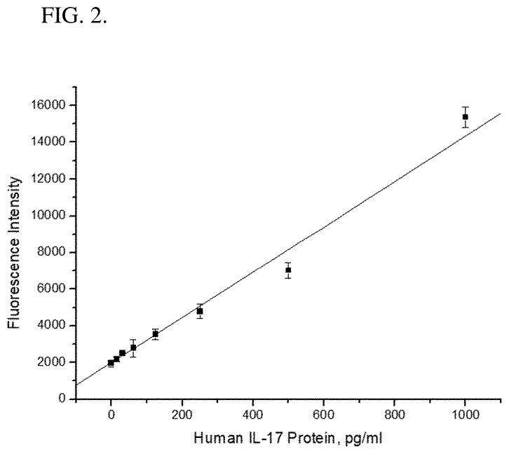

The Aβ microarray was constructed for Quantitative analysis of human IL-17. To construct IL-17 detection chip, capture IL-17 antibody(0.1 mg/ml) was immobilized on each IPS-CHIP plate. IL-17 detection chip was interacted with IL-17 protein in different concentrations ranged from 0.06 to 60000 pg/ml, and then detected by detection IL-17 antibody and Cy5 labeled streptavidin (FIG. 2). IPS-CHIP coated with IPS-Linker was able to detect a low level (0.06 pg/ml) of human IL-17 protein compared to 96well plate-based ELISA (15.6 pg/ml). Taken together, this finding demonstrated that IPS-Linker-coated chip was more sensitive than 96well plate-based ELISA in terms of detecting proteins.

### Example 5. Protein-Protein Interaction Assay for IL-23 and its Receptor by Using IPS-Linker-Coated Chip

100 μg/ml recombinant human IL-23 receptor Fc chimera protein (R&D systems, USA) was immobilized on the IPS-CHIP with immobilization buffer. After rinsing with PBST and DW, each well was blocked with 3% BSA in PBS. The blocked IPS-CHIP was rinsed with PBST and DW, and then dried under a N₂ stream. After the chip was completely dried, IL-23 ligand labeled with Cy5, which was diluted in PBS containing 1% BSA and 30% glycerol with various concentrations, was spotted on the chip, and incubated for 1 hr in a humidity chamber at 37° C. Following rinsing with PBST and DW and drying under N₂ stream, the fluorescence intensity was measured to confirm protein-protein interaction.

Detection and Data Analysis:

The chip was scanned by using a GenePix 4000B scanner (Molecular Devices, USA) and saved as TIFF files. The scanned images were analyzed using a GenePix Pro 6.0 (Molecular Devices, USA), and the data analyzed with Excel (Microsoft, WA) and Origin 6.1 (Originlab, USA).

Results:

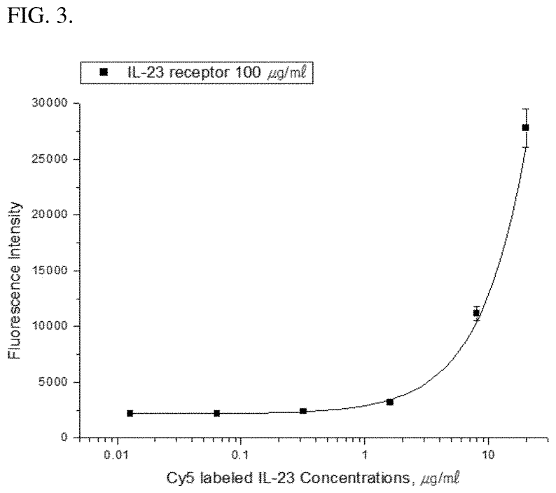

As described previous, 1 μl of protein is spotted in a well. On the each well, 100 ng of recombinant human IL-23 receptor Fc chimera was immobilized, and incubated with IL-23 ligands with various concentrations (12.8 pg to 20 ng) respectively (FIG. 3). As shown in the figure, IL-23 receptors were bound with IL-23 ligands in a dose-dependent manner, and only 1.6 ng of IL-23 ligands was enough for detection. Taken together, IPS-CHIP coated with IPS-Linker is sensitive and cost-effective way to detect protein-protein interaction

### Example 6. Protein-Protein Interaction Assay for PD-1 and PD-L1 by Using IPS-Linker-Coated Chip

Recombinant human PD-1 Fc chimera protein (ACRO Biosystems, USA) was diluted to a working concentration of 1.6 μg/mL to 400 μg/mL in PBS solution with 30% glycerol. PD-1 protein was immobilized on IPS-Linker-coated chip for 24 hr at 4° C. It was then washed twice with 50 mL of PBST solution (10 mM PBS with 0.1% Tween 20, pH 7.8), and dried under a stream surface. The chip was blocked with 0.005% Tween 20 and 3% BSA in PBS solution for 1 hr at room temperature. It was washed in the same way as in the previous procedure. Biotinylated recombinant human PD-L1 protein (R&D Systems, USA) was diluted to a working concentration of 0.4 μg/mL to 100 μg/mL in PBS solution with 30% glycerol, and it was added onto each spot of PD-1 protein on a IPS-CHIP, and incubated for 1 hr at 37° C. Then It was washed in the same way as in the previous procedure. 10 μg/mL of Cy5 conjugated streptavidin (GE Healthcare, USA) was added onto each spot, and incubated for 1 hr at 37° C. It was washed in the same way as in the previous procedure.

Detection and Data Analysis:

The chip was scanned using a GenePix 4000B scanner (Molecular Devices, USA) and saved as TIFF files. The scanned images were analyzed using a GenePix Pro 6.0 (Molecular Devices, USA), and the data analyzed with Excel (Microsoft, WA) and Origin 6.1 (Originlab, USA).

Results:

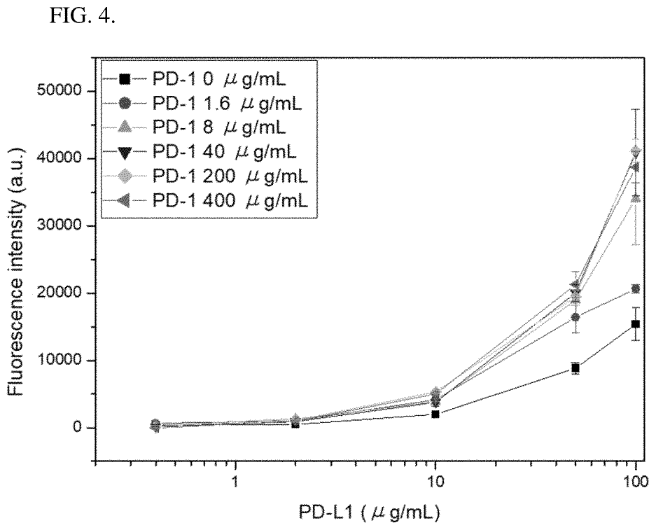

The PD-1 protein in different concentration ranged from 1.6 μg/mL to 400 g/mL was interacted with PD-L1 protein in different concentration ranged from 0.4 g/mL to 100 μg/mL (FIG. 4). It was shown that PD-1 and PD-L1 protein was interacted in a dose-dependent manner. As shown in the figure, the sensitivity of detection limit was 1.6 μg/mL of PD-1 and 10 μg/mL of PD-L1 by using IPS-Linker-coated chip.

### Example 7. Protein-Nucleotide Interaction Assay for STING and c-Di-GMP Binding by Using IPS-Linker-Coated Chip

For concentration-dependent binding analysis between STING proteins and its known ligand, c-di-GMP, His×6 Antibody (Thermo Fisher Scientific, USA) diluted in 30% glycerol at a concentration of 100 μg/ml was immobilized on the IPS-CHIP for 3 hr at 4° C. The chip was washed with PBST (10 mM PBS with 0.1% Tween 20, pH 7.8), and dried with nitrogen gas. Then, the recombinant proteins for His-tagged STING (Active motif, USA) in 30% glycerol was dispensed onto each well of the chip with a series of concentrations of 0 μM, 5 μM, 10 μM, and the reaction was proceeded for overnight at 4° C. Then, the chip was washed with PBST, and dried using nitrogen gas. After blocking for 1 hr at room temperature using 5% BSA, the chip was washed with PBST and dried using nitrogen gas. To test binding capability of STING proteins to its ligand, 2′[DY-547]-AHC-c-di GMP (BioLog, Germany) known as CDN ligand, 2′[DY-547]-AHC-c-di GMP in 30% glycerol was dispensed onto each well on the chip with a series of concentrations of 0 μM, 3.9 μM, 15.62 μM, 62.25 μM, 250 μM, and incubated for 1 hr at 37° C. The chip was washed with PBST, and dried using nitrogen gas.

Detection and Data Analysis:

The chip was scanned using a GenePix 4000B scanner (Molecular Devices, USA) and saved as TIFF files. The scanned images were analyzed using a GenePix Pro 6.0 (Molecular Devices, USA), and the data analyzed with Excel (Microsoft, WA) and Origin 6.1 (Originlab, USA).

Results:

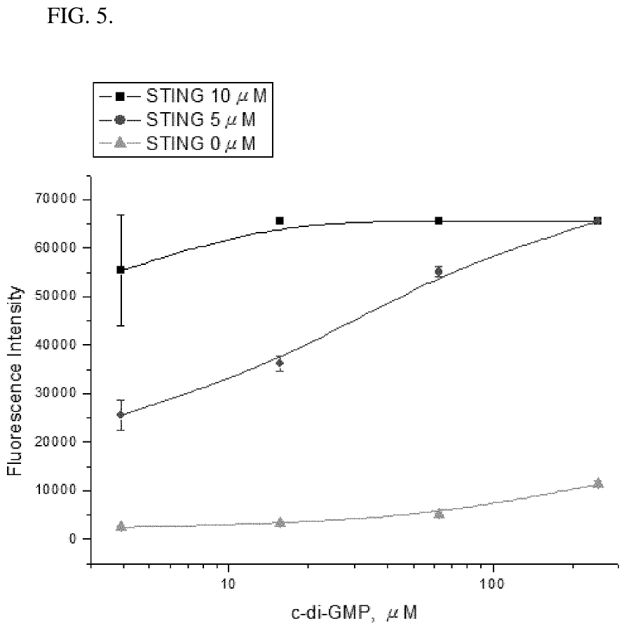

The STING proteins ranged from 5 μM to 10 μM was shown to interact with c-di-GMP ranged from 3.9 μM to 250 μM (FIG. 5) on the chip. Also, the interaction between STING and c-di-GMP was a dose-dependent manner. Taken together, As shown in the figure, IPS-Linker-coated chip was applicable to detect the interaction between proteins (STING) and nucleotide (c-di-GMP) as little as 5 μM of STING proteins and 3.9 μM of c-di-GMP.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1. A calixcrown having Formula I, II, or III: 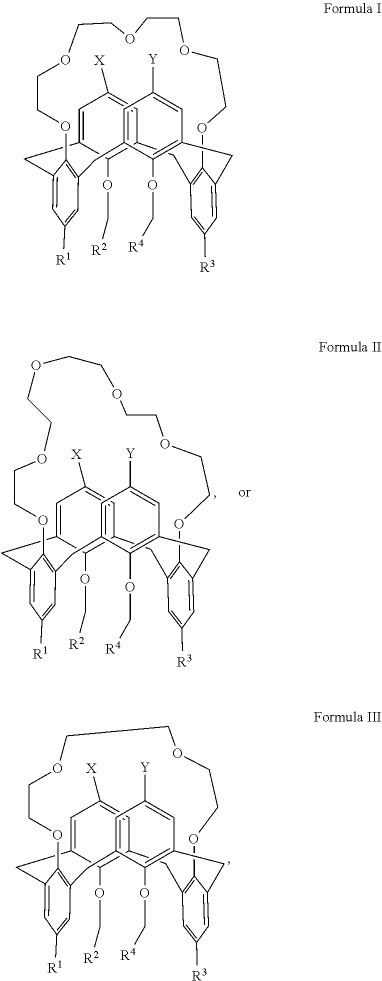
or a salt or ester thereof,
wherein
R¹ and R³ independently represent hydrogen, —CH₂SH, —CH₂Cl, —CH₂CN, —CH₂CHO, CH₂NH₂, or —CH₂COOH;
R² and R⁴ independently represents —CH₂SH, —CH₂Cl, —CH₂CN, —CH₂CHO, —CH₂NH₂, —CH₂COOH, —CN, —CHO, or —COOH; and
X and Y independently represent hydrogen, C₁₋₄ alkyl, OH, or C₁₋₄ alkoxy.

2. The calixcrown of claim 1, or salt or ester thereof, wherein R¹ and R³ are both hydrogen.

3. The calixcrown of claim 1, or salt or ester thereof, wherein X and Y are both hydrogen.

4. The calixcrown of claim 1, or salt or ester thereof, wherein R² and R⁴ are both —COOH.

5. The calixcrown of claim 1, or salt or ester thereof, wherein the calixcrown is characterized by the following formula:

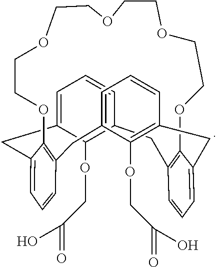 [SMILES: O=C(O)COc1c2cccc1Cc1cccc3c1OCCOCCOCCOCCOc1c(cccc1C2)CC1=CC=CC(C3)C1OCC(=O)O]

6. The calixcrown of claim 1, or salt or ester thereof, wherein the calixcrown is characterized by IPS-Linker A or IPS-Linker B:

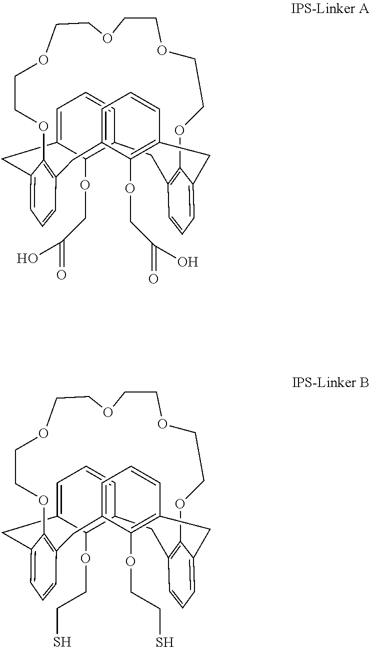 [SMILES: O=C(O)COc1c2cccc1Cc1cccc3c1OCCOCCOCCOCCOc1c(cccc1C2)CC1=CC=CC(C3)C1OCC(=O)O ; SCCOc1c2cccc1Cc1cccc3c1OCCOCCOCCOCCOc1c(cccc1C2)CC1=CC=CC(C3)C1OCCS]

7. A method of immobilizing a protein on a solid substrate, the method comprising:
a) applying the calixcrown of claim 1 onto an inorganic or organic solid substrate to form a calixcrown coated solid substrate;
b) immersing the calixcrown coated solid substrate into a solution comprising the protein.

8. The method of claim 7, wherein the solid substrate is an inorganic solid substrate.

9. The method of claim 8, wherein the solid substrate is a metal solid substrate.

10. The method of claim 7, wherein the solid substrate is selected from the group consisting of gold, silver, glass, Quartz crystal, mica, silicon, polystyrene, and polycarbonate.

11. The method of claim 7, wherein the solution comprises the protein in a concentration of about 1 nM to about 500 uM.

12. The method of claim 7, wherein the protein is selected from Aβ₁₋₄₂, antibodies, enzymes, membrane-bound receptors and non-membrane bound receptors, protein domains and motifs, and intracellular signaling proteins.

13. The method of claim 7, wherein the calixcrown forms a monolayer on the solid substrate.

14. The method of claim 7, wherein the solid substrate is a protein chip, diagnostic kit or protein separation pack.

15. A solid substrate with an immobilized protein prepared by the method of claim 7.

16. A solid substrate coated with the calixcrown of claim 1.

17. The solid substrate of claim 16, wherein the calixcrown forms a monolayer on the solid substrate.

18. The solid substrate of claim 16, wherein the solid substrate is an inorganic solid substrate.

19. The solid substrate of claim 18, wherein the solid substrate is a metal solid substrate.

20. The solid substrate of claim 16, wherein the solid substrate is selected from the group consisting of gold, silver, glass, Quartz crystal, mica, silicon, polystyrene, and polycarbonate.

21. A method of detecting a protein-protein interaction, comprising:
a) immobilizing a first protein on the solid substrate of claim 6 to form a solid substrate with immobilized first protein;
b) incubating the solid substrate with immobilized first protein with a solution comprising a second protein; and
c) detecting an interaction between the immobilized first protein and the second protein.

22. The method of claim 21, wherein the detecting comprises contacting an antibody for the second protein with the solid substrate.

23. The method of claim 22, wherein the detecting further comprises contacting a dye-labeled secondary antibody with the solid substrate, wherein the secondary antibody binds to the antibody for the second protein.

24. The method of claim 23, wherein the detecting further comprises measuring a level of the dye-labeled secondary antibody associated with the solid substrate.

25. The method of claim 21, wherein the solution comprising the second protein derives from a biological fluid sample from a patient with a concentration of about 1 aM (atto M) to about 100 uM.

26. The method of claim 21, wherein the second protein is a biomarker for a cancer, inflammatory disease, neurodegenerative disease, metabolic disease, allergic disease, autoimmune disease, infectious disease, or endocrine disease.

27. The method of claim 21, wherein the second protein is VEGF₁₆₅, antibodies, enzymes, membrane-bound receptors and non-membrane bound receptors, t protein domains and motifs, or intracellular signaling proteins.

28. The method of claim 21, wherein the second protein is IL-17, IL-23, STING, or PD-1.
